(19) United States
(12) Patent Application Publication
(10) Pub. No.: US 2026/0123899 A1
(43) Pub. Date: May 7, 2026

(54) PHOTON-COUNTING-BASED HYBRID FLAT PANEL DETECTOR, IMAGE DATA READING METHOD, AND MEDICAL IMAGING APPARATUS

(71) Applicant: Shanghai United Imaging Healthcare Co., Ltd., Shanghai (CN)

(72) Inventors: Jun Xiang, Shanghai (CN); Canzhen Ma, Shanghai (CN); Jianqing Sun, Shanghai (CN); Hui Yin, Shanghai (CN)

(21) Appl. No.: 19/430,169

(22) Filed: Dec. 22, 2025

Related U.S. Application Data
(63) Continuation-in-part of application No. PCT/CN2024/100774, filed on Jun. 21, 2024.

(30) Foreign Application Priority Data
Jun. 21, 2023 (CN) 202310750080.1

(51) Int. Cl. A61B 6/42; A61B 6/00; A61B 6/40

(52) U.S. Cl. CPC A61B 6/4241; A61B 6/4014; A61B 6/4078; A61B 6/4441

(57) ABSTRACT

Disclosed is a photon-counting-based hybrid flat panel detector. The photon-counting-based hybrid flat panel detector includes a photon-counting detection module having a first radiation detection surface, and two energy-integrating detection modules each having a second radiation detection surface. The first radiation detection surface extends in a strip shape along a first direction, and the second radiation detection surfaces are planar. The two energy-integrating detection modules are arranged on two opposite sides of the photon-counting detection module, such that the second radiation detection surfaces are arranged on two opposite sides of the first radiation detection surface in a second direction. The first radiation detection surface and the second radiation detection surfaces jointly form a radiation detection surface of the photon-counting-based hybrid flat panel detector. Also disclosed are a medical imaging apparatus and an image data reading method.

### CROSS-REFERENCE TO RELATED APPLICATIONS

The present application is a continuation-in-part application of international patent application No. PCT/CN2024/100774, filed on Jun. 21, 2024, which claims priority to Chinese patent application No. 202310750080.1, filed on Jun. 21, 2023. The contents of the above identified applications are hereby incorporated herein in their entireties by reference.

### TECHNICAL FIELD

The present disclosure relates to the technical field of medical imaging apparatus, and in particular, to a photon-counting-based hybrid flat panel detector, an image data reading method, and a medical imaging apparatus.

### BACKGROUND

As a solid-state X-ray digital radiography device, a Flat Panel Detector (FPD) is often used in the medical field, such as in Digital Radiography (DR) systems, Full-Field Digital Mammography (FFDM) systems, Cone Beam Computed Tomography (CBCT) systems, and the like. Traditional FPDs include energy-integrating flat panel detectors, hereinafter referred to as Energy Integrating Detectors (EIDs). Due to the advantages of a simple manufacturing process, a low cost, and a large field of view, EIDs are widely used in medical imaging systems (such as X-ray imaging) and can perform real-time planar radiography, such as fluoroscopy, digital subtraction angiography, or cone-beam CT, to facilitate better formulation of treatment plans, monitoring, and evaluation.

However, EIDs cannot provide a reliable capability to resolve low-contrast anatomical features required for detecting low-contrast lesions. EIDs also lack high-resolution capability for visualizing fine structures such as stent kinks and narrowing, as well as the spectral and quantitative imaging capabilities that are highly needed by doctors. The main reasons lie in the flaws in the detection principle of EID hardware. For example, the analog-to-digital converters have a low number of quantization bits, scintillator materials have afterglow and related hysteresis effects, the charge mobility in the amorphous silicon-based thin-film transistor array is poor, and pixel design related to these factors.

Semiconductor-based Photon Counting Detectors (PCDs) have multiple advantages over EIDs. These advantages include, but are not limited to, the following: the energy threshold of PCDs can almost eliminate electronic noise, the detector hysteresis effect of PCDs is negligible, and PCDs can achieve low-contrast resolution through favorable X-ray photon energy weighting. Compared with the pixel binning required in EID data acquisition with scintillators, PCDs have higher spatial resolution. PCDs can obtain multi-energy-level spectral information.

Nevertheless, PCDs are costly, and the cost increases linearly with the increase in the X-ray sensing area. The high-cost solution of adopting photon-counting detection technology for the entire panel hinders clinical promotion and application. Large-area PCDs have uneven responses. In low-contrast scenarios, X-ray scattering has a non-negligible negative impact on the images captured by large-area PCDs.

### SUMMARY

In a first aspect of the present disclosure, a photon-counting-based hybrid flat panel detector is provided. The photon-counting-based hybrid flat panel detector includes a photon-counting detection module and two energy-integrating detection modules. The photon-counting detection module has a first radiation detection surface. The first radiation detection surface extends in a strip shape along a first direction. Each of the two energy-integrating detection modules has a planar second radiation detection surface. The two energy-integrating detection modules are arranged on opposite two sides of the photon-counting detection module along a second direction, so that the second radiation detection surfaces are provided on the opposite two sides of the first radiation detection surface along the second direction. The second direction is parallel to the first radiation detection surface and perpendicular to the first direction. The first radiation detection surface and the second radiation detection surfaces jointly form the radiation detection surface of the photon-counting-based hybrid flat panel detector.

In the first aspect, the first radiation detection surface is located at a central position of a radiation detection surface of the photon-counting-based hybrid flat panel detector along the second direction.

In the first aspect, the first radiation detection surface penetrates the radiation detection surface of the photon-counting-based hybrid flat panel detector along the first direction.

In the first aspect, the first radiation detection surface is coplanar with the second radiation detection surface of each of the two energy-integrating detection modules.

In the first aspect, the second radiation detection surface of one of the two energy-integrating detection modules is coplanar with the second radiation detection surface of the other one of the two energy-integrating detection modules, and is not coplanar with the first radiation detection surface.

In the first aspect, an edge of the second radiation detection surface close to the first radiation detection surface is arc-shaped.

In the first aspect, the photon-counting detection module includes a main photon detection circuit and at least one photon-counting detection unit. The photon-counting detection unit is electrically connected to the main photon detection circuit, and the photon-counting detection unit has a reference photon-counting detection surface.

In the first aspect, the at least one photon-counting detection unit includes a plurality of photon-counting detection units arranged in at least one row. Each of the plurality of photon-counting detection units has a reference photon-counting detection surface, and all the reference photon-counting detection surfaces are coplanar to form the first radiation detection surface.

In the first aspect, the plurality of photon-counting detection units is arranged along the first direction.

In the first aspect, the photon-counting detection unit includes a photoelectric conversion substrate, an electrical signal acquisition substrate, and a photon-counting detection circuit. An end surface of the photoelectric conversion substrate is configured as the reference photon-counting detection surface. The electrical signal acquisition substrate is electrically connected to the photoelectric conversion substrate. The photon-counting detection circuit is electrically connected to the electrical signal acquisition substrate and the main photon detection circuit.

In the first aspect, each of the two energy-integrating detection modules includes an energy-integrating substrate and an energy-integrating detection circuit electrically connected to the energy-integrating substrate. An end surface of the energy-integrating substrate is configured as the second radiation detection surface.

In the first aspect, the first radiation detection surface of the photon-counting detection module and the second radiation detection surfaces of the two energy-integrating detection modules are spliced together to form the radiation detection surface of the photon-counting-based hybrid flat panel detector.

In the first aspect, each of the first radiation detection surface and the second radiation detection surfaces has a pixel array, and each pixel array includes a plurality of uniformly arranged pixels. For the adjacent first radiation detection surface and second radiation detection surface, a splicing gap along the second direction between the column of pixels of the first radiation detection surface closest to the second radiation detection surface and the column of pixels of the second radiation detection surface closest to the first radiation detection surface is less than or equal to the size of two pixels.

In the first aspect, the splicing gap is equal to the size of one pixel.

In the first aspect, the area of the second radiation detection surface is larger than that of the first radiation detection surface.

In a second aspect of the present disclosure, an image data reading method is provided, applied to the photon-counting-based hybrid flat panel detector according to the first aspect. The first radiation detection surface includes a plurality of reference photon counting detection surfaces arranged in sequence along the first direction. The two energy-integrating detection modules include a first energy-integrating detection module and a second energy-integrating detection module. Each of the second radiation detection surfaces includes a plurality of rows of reference energy-integrating detection surfaces arranged in sequence along the second direction. The image data reading method includes: reading image data of the first energy-integrating detection module, the photon-counting detection module, and the second energy-integrating detection module in sequence along the second direction; or reading the image data of the first energy-integrating detection module, the photon-counting detection module, and the second energy-integrating detection module synchronously. In the second aspect, reading the image data of the photon-counting detection module includes: reading the image data of the photon-counting detection module in a first data reading mode. The first data reading mode is configured to read the image data of the reference photon counting detection surfaces in sequence along the first direction.

In the second aspect, reading the image data of the first energy-integrating detection module, the photon-counting detection module, and the second energy-integrating detection module in sequence along the second direction includes: reading the image data of the first energy-integrating detection module in a second data reading mode; reading the image data of the second energy-integrating detection module in a third data reading mode. The second data reading mode is configured to read the image data of reference energy-integrating detection surfaces in the energy-integrating detection module row by row in a direction along the second direction and close to the photon counting detection module. The third data reading mode is configured to read the image data of reference energy-integrating detection surfaces in the energy-integrating detection module row by row in a direction along the second direction and away from the photon counting detection module.

In the second aspect, reading the image data of the first energy-integrating detection module, the photon counting detection module, and the second energy-integrating detection module synchronously includes: reading the image data of the first energy-integrating detection module and the second energy-integrating detection module synchronously in the second data reading mode or the third data reading mode. The second data reading mode is configured to read the image data of the reference energy-integrating detection surfaces in the energy-integrating detection module row by row in a direction along the second direction and close to the photon counting detection module. The third data reading mode is configured to read the image data of the reference energy-integrating detection surfaces in the energy-integrating detection module row by row in a direction along the second direction and away from the photon counting detection module.

In a third aspect of the present disclosure, a non-transitory computer-readable storage medium is provided, storing a program executable by a processor. The processor is configured to read image data from the photon-counting-based hybrid flat panel detector according to the first aspect. The first radiation detection surface includes a plurality of reference photon counting detection surfaces arranged in sequence along the first direction. The two energy-integrating detection modules include a first energy-integrating detection module and a second energy-integrating detection module. Each of the second radiation detection surfaces includes a plurality of rows of reference energy-integrating detection surfaces arranged in sequence along the second direction. When the program is executed by the processor, the processor implements reading the image data of the first energy-integrating detection module, the photon counting detection module, and the second energy-integrating detection module in sequence along the second direction; or reading the image data of the first energy-integrating detection module, the photon counting detection module, and the second energy-integrating detection module synchronously.

In a fourth aspect of the present disclosure, a medical imaging apparatus is provided, including a C-shaped arm having opposite two ends; a radiation source disposed on one of two ends of the C-shaped arm; a collimator disposed on the radiation source to limit the radiation emitted by the radiation source to a beam of a predetermined shape; and the photon-counting-based hybrid flat panel detector according to the above first aspect, disposed on the other one of the two ends of the C-shaped arm. The first radiation detection surface and the second radiation detection surfaces face the radiation source to receive the beam.

In the fourth aspect, the collimator is configured to limit the radiation emitted by the radiation source to a fan beam. The first radiation detection surface of the photon-counting-based hybrid flat panel detector is arranged to be suitable for receiving the radiation of the fan beam. The extending direction of the first radiation detection surface is parallel to the fan beam.

### BRIEF DESCRIPTION OF DRAWINGS

To more clearly illustrate the technical solutions in the embodiments of the present disclosure or the conventional technologies, the drawings needed in the description of the embodiments or the conventional technologies will be briefly introduced below. Obviously, the drawings in the following description involve only some embodiments of the present disclosure. For those of ordinary skill in the art, other drawings can be obtained according to these drawings without inventive efforts.

FIG. 1 is a schematic side view of a photon-counting-based hybrid flat panel detector according to an embodiment of the present disclosure.

FIG. 2 is a schematic side view of a photon-counting-based hybrid flat panel detector according to another embodiment of the present disclosure.

FIG. 3 is a schematic top view of a photon-counting-based hybrid flat panel detector according to an embodiment of the present disclosure.

FIG. 4 is another schematic top view of a photon-counting-based hybrid flat panel detector according to an embodiment of the present disclosure.

FIG. 5 is yet another schematic top view of a photon-counting-based hybrid flat panel detector according to an embodiment of the present disclosure.

FIG. 6 is a schematic view showing that a second radiation detection surface of an energy-integrating detection module is arc-shaped according to an embodiment of the present disclosure.

FIG. 7 is a schematic view of a photon-counting detection module and an energy-integrating detection module of a photon-counting-based hybrid flat panel detector according to an embodiment of the present disclosure.

FIG. 8 is a schematic view of a photon-counting detection module according to an embodiment of the present disclosure.

FIG. 9 is another schematic view of a photon-counting detection module according to an embodiment of the present disclosure.

FIG. 10 is a schematic view of a splicing of a photon-counting detection module and an energy-integrating detection module according to an embodiment of the present disclosure.

FIG. 11 is a schematic view of a data reading method of a photon-counting-based hybrid flat panel detector according to an embodiment of the present disclosure.

FIG. 12 is a schematic view of a data reading method of a photon-counting-based hybrid flat panel detector according to another embodiment of the present disclosure.

FIG. 13 is a schematic view of a medical imaging apparatus according to an embodiment of the present disclosure.

FIG. 14 is another schematic view of a medical imaging apparatus according to an embodiment of the present disclosure.

FIG. 15 is a schematic view of a medical imaging apparatus performing a 2D full-frame imaging mode according to an embodiment of the present disclosure.

FIG. 16 is a schematic view of a medical imaging apparatus performing a 2D strip imaging mode according to an embodiment of the present disclosure.

FIG. 17 is a schematic view of a medical imaging apparatus performing a 3D full-frame imaging mode according to an embodiment of the present disclosure.

FIG. 18 is a schematic view of a medical imaging apparatus performing a 3D strip imaging mode according to an embodiment of the present disclosure.

### DETAILED DESCRIPTION

To make the purposes, advantages, and features of the present disclosure clearer, the present disclosure is further described in detail below in conjunction with the accompanying drawings and specific embodiments. It should be noted that the accompanying drawings are all in a very simplified form and are not drawn to scale, and are only used to conveniently and clearly assist in explaining the purposes of the embodiments of the present disclosure. In addition, the structures shown in the drawings are often part of the actual structures. In particular, the drawings need to show different focuses and may adopt different scales.

As used herein, the singular forms “a”, “an”, and “the” include plural referents. The term “or” is usually used in the sense of “and/or”. The term “several” is usually used in the sense of “at least one”. The term “at least two” is usually used in the sense of “two or more”. In addition, the terms “first”, “second”, and “third” are only used for descriptive purposes and cannot be understood as indicating or implying relative importance or implicitly indicating the number of the indicated technical features. Thus, the features defined with “first”, “second”, and “third” may explicitly or implicitly include one or at least two such features. “An end” and “the other end”, as well as “proximal end” and “distal end”, usually refer to a corresponding two parts, which include not only the endpoints. The terms “mounting”, “connecting”, and “connection” should be understood in a broad sense. For example, the connection may be a fixed connection, a detachable connection, or an integrated connection. The connection may be a mechanical connection or an electrical connection. The connection may be a direct connection or an indirect connection through an intermediate medium, and may be an internal communication between two elements or an interaction relationship between the two elements. In addition, as used herein, when one element is disposed on another element, it usually only indicates that there is a connection, coupling, coordination, or transmission relationship between the two elements, and the two elements may be directly or indirectly connected, coupled, coordinated, or transmitted through an intermediate element. It cannot be understood as indicating or implying the spatial positional relationship between the two elements. That is, one element may be located inside, outside, above, below, or on one side of the other element, etc., unless the content clearly indicates otherwise. For those of ordinary skill in the art, the specific meanings of the above terms in the present disclosure can be understood according to specific situations.

In conventional technologies, there have been hybrid flat panel detectors designed by integrating the imaging technology of PCDs and EIDs. Such hybrid flat panel detectors can combine the advantages of EIDs and PCDs. Conventional hybrid flat panel detectors usually have a PCD stacked on one side of an EID in the vertical direction. However, the detection surface of the PCD is usually a narrow beam, and the volume thereof is relatively large. A displacement device needs to be added to move the PCD before EID image acquisition can be performed, which affects the use of the EID. For hybrid flat panel detectors with a PCD attached next to an EID, the position of the EID also needs to be adjusted as a whole for PCD imaging acquisition to ensure that the imaging results of the two parts do not interfere with each other.

In the imaging acquisition process of the conventional hybrid flat panel detectors, it is necessary to move one of the detectors to perform image acquisition of another detector. The operation is relatively complex, and the two detectors cannot receive rays and image simultaneously. In addition, such stacked hybrid detectors in conventional technologies have a large volume, a heavy weight, and a complex manufacturing process, and the performance of the obtained local images cannot reach the highest level.

To address the problems existing in the conventional hybrid flat panel detectors, a photon-counting-based hybrid flat panel detector is provided in the present disclosure. The photon-counting-based hybrid flat panel detector can obtain high-performance local images at a limited cost, simplify the manufacturing process, reduce the volume, and perform both energy-integrating radiography and photon-counting radiography simultaneously.

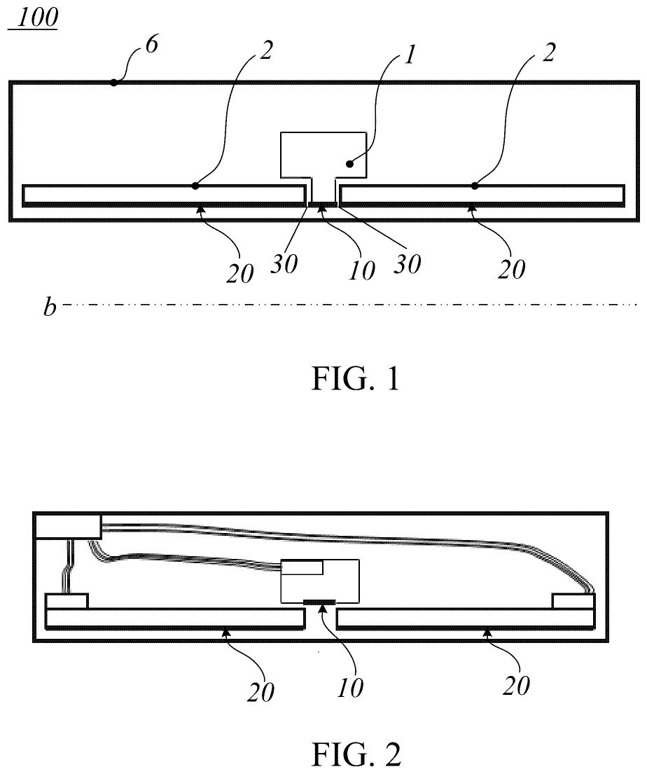

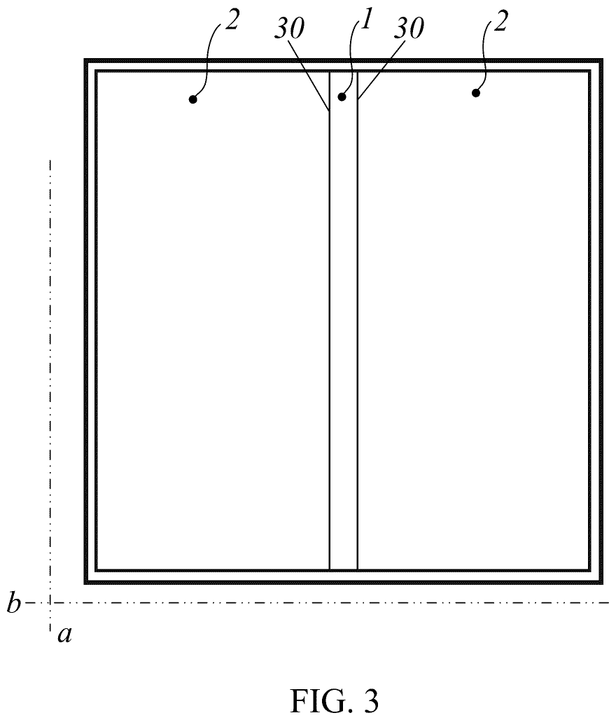

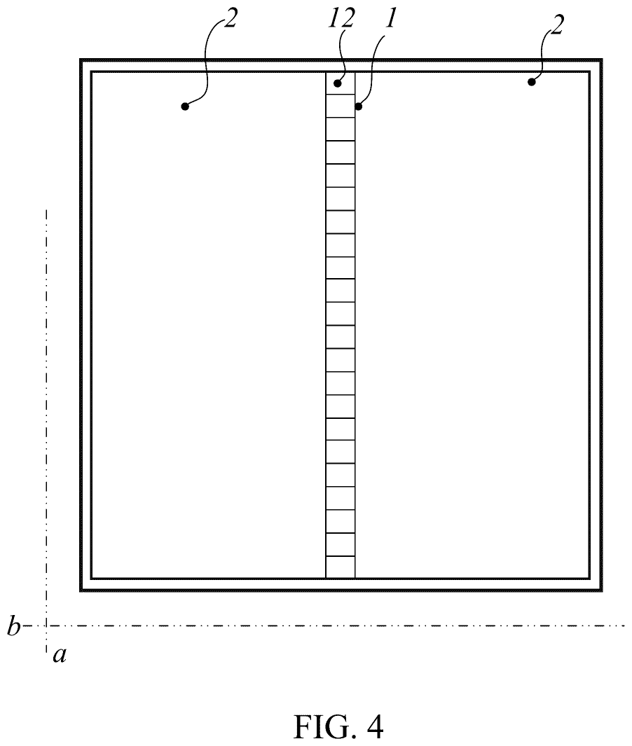

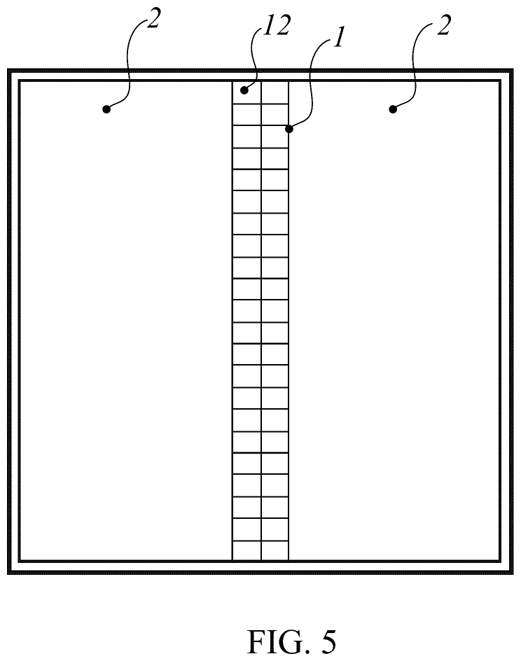

FIG. 1 is a schematic side view of a photon-counting-based hybrid flat panel detector according to an embodiment of the present disclosure. FIG. 2 is a schematic side view of a photon-counting-based hybrid flat panel detector according to another embodiment of the present disclosure. FIG. 3 is a schematic top view of a photon-counting-based hybrid flat panel detector according to an embodiment of the present disclosure. FIG. 4 is another schematic top view of a photon-counting-based hybrid flat panel detector according to an embodiment of the present disclosure. FIG. 5 is yet another schematic top view of a photon-counting-based hybrid flat panel detector according to an embodiment of the present disclosure.

As shown in FIGS. 1 to 5, in an embodiment of the present disclosure, a photon-counting-based hybrid flat panel detector 100 is provided. The hybrid flat panel detector 100 includes a photon-counting detection module 1 and two energy-integrating detection modules 2. The photon-counting detection module 1 has a first radiation detection surface 10, and the first radiation detection surface 10 extends in a strip shape along a first direction a. Referring to FIG. 3, the first radiation detection surface 10 is in an elongated strip shape. The photon-counting detection module 1 may also be in a strip shape, and the first radiation detection surface 10 is located on an end surface of the photon-counting detection module 1. The photon-counting detection module 1 detects radiation through the first radiation detection surface 10 and obtains photon-counting medical images by means of photon counting. When the hybrid flat panel detector 100 receives fan-beam radiation, the photon-counting detection module 1 can receive the complete fan-beam radiation. In this case, the first direction a along which the first radiation detection surface 10 extends corresponds to the length direction of the projection of the fan beam on the radiation detection surface of the hybrid flat panel detector 100, that is, the extending direction of the first radiation detection surface 10 is parallel to the fan beam. The energy-integrating detection module 2 has a second radiation detection surface 20, and the second radiation detection surface 20 is basically planar. In other embodiments, the second radiation detection surface 20 may also have a certain curvature. The energy-integrating detection module 2 detects radiation through the second radiation detection surface 20 and obtains energy-integrating medical images in an energy-integrating manner. Rays are emitted by a radiation source of the medical imaging apparatus. In one embodiment, the radiation source includes, for example, an X-ray tube configured to emit X-rays. The two energy-integrating detection modules 2 are arranged on opposite two sides of the photon-counting detection module 1 along a second direction b, so that the second radiation detection surfaces 20 are provided on both sides of the first radiation detection surface 10 along the second direction b. The second direction b is parallel to the first radiation detection surface 10 and perpendicular to the first direction a. It can be understood that the first direction a is the length direction of the strip-shaped first radiation detection surface 10, and the second direction b is the width direction of the strip-shaped first radiation detection surface 10. The first radiation detection surface 10 and the second radiation detection surfaces 20 jointly form the radiation detection surface of the hybrid flat panel detector 100. The area of the second radiation detection surface 20 is larger than that of the first radiation detection surface 10. For example, the second radiation detection surface 20 has the same length as the first radiation detection surface 10, and the width (dimension along the second direction b) of the second radiation detection surface 20 is larger than that of the first radiation detection surface 10. Referring to FIG. 3, in this embodiment, the widths of the two second radiation detection surfaces 20 along the second direction b are equal, that is, the first radiation detection surface 10 is located at a central position of the radiation detection surface of the hybrid flat panel detector 100 along the second direction b. In this embodiment, the photon-counting detection module 1 is spliced between the two energy-integrating detection modules 2.

Referring to FIG. 3, in this embodiment, the first radiation detection surface 10 penetrates the detection surface of the hybrid flat panel detector 100 along the first direction a to separate the two second radiation detection surfaces 20 along the second direction b. As a result, the two second radiation detection surfaces 20 are only distributed on one of the opposite two sides of the first radiation detection surface 10, respectively, on the detection surface of the hybrid flat panel detector 100, that is, on the entire detection surface of the hybrid flat panel detector 100, the two second radiation detection surfaces 20 are distributed discontinuously. The first radiation detection surface 10 is an elongated rectangle with a constant width along the first direction a.

The hybrid flat panel detector 100 provided in the present disclosure is provided with two energy-integrating detection modules 2 and a photon-counting detection module. The photon-counting detection module 1 is arranged between the two energy-integrating detection modules 2. As such, the hybrid flat panel detector 100 of this embodiment can obtain medical images with no electronic noise, high spatial resolution, and multi-energy-level image information based on the photon-counting detection module 1, and can obtain large-area medical images based on the energy-integrating detection modules 2 which are low-cost and widely used. The hybrid flat panel detector 100, combining photon counting and energy integration, has many advantages. At a limited cost, the photon-counting detection module can be used locally, which can reduce local electronic noise, improve contrast, enhance spatial resolution, and realize multi-energy-level imaging. After reducing the performance of the photon-counting detection module 1, the photon-counting detection module can be compatible with the widely used energy-integrating detection modules 2. In addition, compared with the conventional hybrid flat panel detector where the PCD is stacked on the EID, the hybrid flat panel detector 100 provided in the present disclosure has only two splicing seams by arranging the photon-counting detection module 1 between the two energy-integrating detection modules 2, which reduces the impact of the splicing seams on imaging.

The hybrid flat panel detector provided in the present disclosure realizes a combination of the first radiation detection surface and the second radiation detection surfaces by arranging the photon-counting detection module between the two energy-integrating detection modules, thereby realizing a combination of two radiography methods, namely energy-integrating imaging and photon-counting imaging. Compared with the conventional energy-integrating flat panel detector, the hybrid flat panel detector provided in the present disclosure uses the photon-counting detection module locally at a limited cost. For a generated full-frame image, the electronic noise can be reduced locally, the contrast can be improved, the spatial resolution can be enhanced, and the multi-energy-level imaging can be achieved.

In an embodiment, a strip-shaped photon-counting detection module can be corresponding to the position of a region of interest, so as to obtain more information about the region of interest. The hybrid mode where the strip-shaped first radiation detection surface is arranged between the two second radiation detection surfaces can realize strip-shaped photon-counting detection in a single direction by using a photon-counting detection module with a smaller area, thereby generating a full-frame image with local photon-counting information. For 2D imaging, the hybrid flat panel detector with strip-shaped high-performance information can provide noise reduction, enhancement, or other effects for the information around the strip and even the entire detector through conventional image processing or artificial intelligence algorithms. Compared with a local square distributed hybrid detection method, such a configuration can reduce the image processing to a one-dimensional method for local strip performance reduction, full-frame noise reduction or full-frame enhancement, or other processing.

Although, the above embodiment describes the hybrid flat panel detector 100 including one photon-counting detection module 1 and two energy-integrating detection modules 2, in other embodiments, the hybrid flat panel detector 100 can include more than one photon-counting detection module 1 and more than two energy-integrating detection modules 2 arranged adjacent to the photon-counting detection modules 1.

In some embodiments, the first radiation detection surface 10 and the two second radiation detection surfaces 20 are coplanar (as shown in FIG. 1). In this case, the scattered radiation received can be minimized. In other embodiments, the first radiation detection surface 10 is not coplanar with the second radiation detection surfaces 20 (as shown in FIG. 2), that is, the two second radiation detection surfaces 20 are not coplanar with the first radiation detection surface 10, but the two second radiation detection surfaces 20 are coplanar.

As shown in FIG. 1, when the photon-counting detection module 1 and the energy-integrating detection modules 2 are spliced together and the first radiation detection surface 10 is coplanar with the second radiation detection surfaces 20, there are splicing seams 30 between the first radiation detection surface 10 and the second radiation detection surfaces 20 along the second direction b. A width of the splicing seams can be adjusted by mechanical adjustment. Referring to the embodiment shown in FIG. 3, when the first radiation detection surface 10 is spliced with the two second radiation detection surfaces 20 to form the radiation detection surface of the hybrid flat panel detector, there are only two splicing seams 30 on the radiation detection surface of the hybrid flat panel detector. Specifically, along the first direction a, each splicing seam 30 penetrates the detection surface of the hybrid flat panel detector. In this embodiment, the opposite two sides of the strip-shaped first radiation detection surface 10 of the photon-counting detection module 1 along the second direction b are spliced with the second radiation detection surfaces 20 of the two energy-integrating detection modules 2, respectively. The splicing of the photon-counting detection module 1 and the energy-integrating detection modules 2 through such a splicing method can simplify the manufacturing of the hybrid flat panel detector.

As shown in FIG. 2, the photon-counting detection module 1 is located behind the two energy-integrating detection modules 2 and spliced with the two energy-integrating detection modules 2. The first radiation detection surface 10 is not coplanar with the two second radiation detection surfaces 20, while the two second radiation detection surfaces 20 are coplanar. In this embodiment, a projection of the first radiation detection surface 10 on a plane where the two second radiation detection surfaces 20 are located is between the two second radiation detection surfaces 20, and occupies the gap between the two second radiation detection surfaces 20 without overlapping with the two second radiation detection surfaces 20. In other embodiments, opposite two edge regions of a projection of the first radiation detection surface 10 on a plane where the two second radiation detection surfaces 20 are located may overlap with the two second radiation detection surfaces 20.

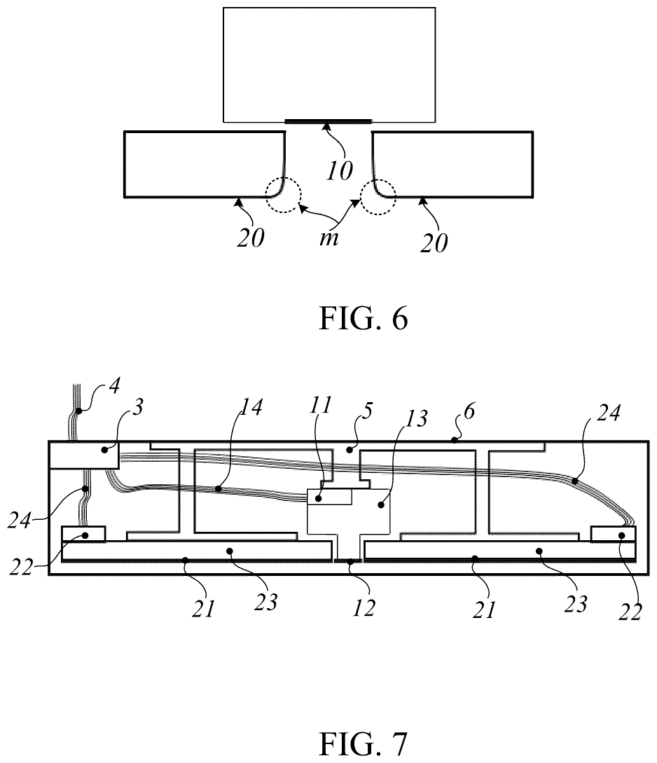

In some embodiments, when the first radiation detection surface 10 is not coplanar with the two second radiation detection surfaces 20 and the two second radiation detection surfaces 20 are coplanar, an edge of each second radiation detection surface 20 close to the first radiation detection surface 10 is arc-shaped (as shown at m in FIG. 6). In this case, both the arc-shaped edge of the second radiation detection surfaces 20 and a side surface of the energy-integrating detection modules 2 close to the first radiation detection surface 10 can receive radiation. Therefore, an effective detection area of the second radiation detection surface 20 for radiation can be increased, more radiation can be detected, and thereby higher-quality images can be obtained.

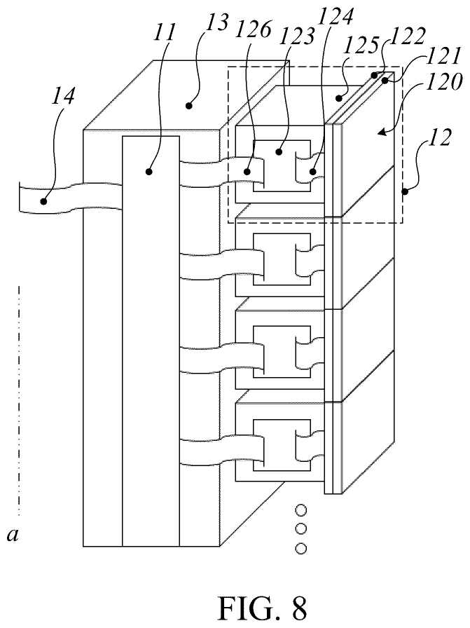

Referring to FIG. 7, the hybrid flat panel detector 100 further includes a hybrid detection circuit 3, a hybrid detection interface 4, a hybrid mechanical frame 5, and a hybrid housing 6. The hybrid detection circuit 3, the hybrid mechanical frame 5, the photon-counting detection module 1, and the energy-integrating detection modules 2 are all located in the hybrid housing 6. The hybrid mechanical frame 5 is fixed on an inner wall of the hybrid housing 6. The hybrid detection interface 4 is located on an outer wall of the hybrid housing 6. The photon-counting detection module 1 and the energy-integrating detection modules 2 are fixed on the hybrid mechanical frame 5. The photon-counting detection module 1 and the energy-integrating detection modules 2 are communicatively connected to the hybrid detection circuit 3. The hybrid detection circuit 3 realizes information interaction with the outside through the hybrid detection interface 4, including connecting a power supply to supply power to the hybrid flat panel detector 100, controlling radiation detection of the photon-counting detection module 1 and the energy-integrating detection modules 2 through an external controller, and reading the image information of the photon-counting detection module 1 and the energy-integrating detection modules 2.

Referring to FIG. 7, regarding a specific structure of the energy-integrating detection module 2, the energy-integrating detection module 2 includes an energy-integrating substrate 21 and an energy-integrating detection circuit 22 electrically connected to the energy-integrating substrate. An end surface of the energy-integrating substrate 21 is configured as the second radiation detection surface 20. The energy-integrating detection module further includes a second mechanical frame 23 and a second detection interface 24. The second mechanical frame 23 is fixed on the hybrid mechanical frame 5. The energy-integrating substrate 21 is fixed on the second mechanical frame 23. The energy-integrating detection circuit 22 is arranged on the second mechanical frame 23. The energy-integrating detection circuit 22 is electrically connected to the hybrid detection circuit 3. The energy-integrating substrate 21 receives radiation (such as X-rays) and detects the incident dose of radiation in an energy-integrating manner. The energy-integrating detection circuit 22 processes signals related to the incident dose of radiation. The energy-integrating detection circuit 22 is communicatively connected to the hybrid detection circuit 3 through the second detection interface 24, so as to realize information interaction between the energy-integrating detection module 2 and the outside through the hybrid detection circuit 3. The information interaction includes connecting a power supply to supply power to the energy-integrating detection module 2, controlling the radiation detection of the energy-integrating detection module 2 through an external controller, and reading the image information of the energy-integrating detection module 2.

Regarding a specific structure of the photon-counting detection module 1, referring to FIG. 8, the photon-counting detection module 1 includes a main photon detection circuit 11 and at least one photon-counting detection unit 12. The photon-counting detection unit 12 is electrically connected to the main photon detection circuit 11. The photon-counting detection unit 12 has a reference photon-counting detection surface 120. The first radiation detection surface 10 can be formed through the reference photon-counting detection surface 120. The photon-counting detection module 1 further includes a first mechanical frame 13 and a first detection interface 14. The first mechanical frame 13 is fixedly connected to the hybrid mechanical frame 5. The photon-counting detection unit 12 is arranged on the first mechanical frame 13. The main photon detection circuit 11 is arranged on the first mechanical frame 13. The main photon detection circuit 11 is communicatively connected to the hybrid detection circuit 3 through the first detection interface 14. The photon-counting detection unit 12 detects and receives radiation through the reference photon-counting detection surface 120, and detects the incident dose of radiation in a photon-counting manner. The main photon detection circuit 11 processes the signals about the incident dose of radiation from all the photon-counting detection units 12, and realizes information interaction between the photon-counting detection unit 12 and the outside through the hybrid detection circuit 3. The information interaction includes connecting a power supply to supply power to the photon-counting detection unit 12, controlling the radiation detection of the photon-counting detection unit 12 through an external controller, and reading the image information of the photon-counting detection unit 12.

Referring to FIG. 8 in combination with FIGS. 3 to 5, the form of forming the first radiation detection surface 10 through the reference photon-counting detection surfaces 120 is described below. In an embodiment, referring to FIG. 3, a photon-counting detection unit 12 extends in a strip shape so that the reference photon-counting detection surface 120 forms the first radiation detection surface 10. In other words, the photon-counting detection module 1 includes a photon-counting detection unit 12, and the photon-counting detection unit 12 extends in a strip shape, so that the reference photon-counting detection surface 120 of the photon-counting detection unit 12 extends to form the strip-shaped first radiation detection surface 10. In another embodiment, referring to FIGS. 4 and 8, a plurality of photon-counting detection units 12 are arranged in a row in sequence along the first direction a. The photon-counting detection module 1 includes a row of photon-counting detection units 12. As such, the reference photon-counting detection surfaces 120 of each photon-counting detection unit 12 in the row are coplanar to form the first radiation detection surface 10. In yet another embodiment, referring to FIG. 5 and in combination with FIG. 8, the photon-counting detection module 1 includes at least two rows of photon-counting detection units 12 arranged side by side, and the reference photon-counting detection surfaces 120 of all the photon-counting detection units 12 are coplanar to form the first radiation detection surface 10.

Referring to FIG. 8, a specific structure of the photon-counting detection unit 12 is described below. The photon-counting detection unit 12 includes a photoelectric conversion substrate 121, an electrical signal acquisition substrate 122, a photon-counting detection circuit 123, a signal transmission interface 124, a third mechanical frame 125, and a photon-counting data interface 126. An end surface of the photoelectric conversion substrate 121 is configured as the reference photon-counting detection surface 120 capable of detecting radiation. The electrical signal acquisition substrate 122 is arranged parallel to the photoelectric conversion substrate 121. The electrical signal acquisition substrate 122 is electrically connected to the photoelectric conversion substrate 121. The electrical signal acquisition substrate 122 is fixedly connected to the third mechanical frame 125. The third mechanical frame 125 is fixed on the first mechanical frame 13. The photon-counting detection circuit 123 is electrically connected to the electrical signal acquisition substrate 122 through the signal transmission interface 124, and is electrically connected to the main photon detection circuit 11 through the photon-counting data interface 126. The photoelectric conversion substrate 121 directly converts radiation into electrical signals. The electrical signal acquisition substrate 122 collects the electrical signals and transmits them to the photon-counting detection circuit 123 through the signal transmission interface 124. The photon-counting detection circuit 123 processes the electrical signals and then transmits them to the main photon detection circuit 11 through the photon-counting data interface 126.

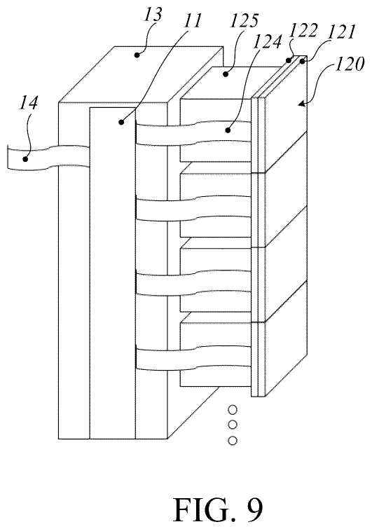

In an embodiment, referring to FIG. 9, the photon-counting detection circuit 123 can be integrated on the main photon detection circuit 11, that is, all the photon-counting detection units 12 share the same main photon detection circuit 11 with the function of the photon-counting detection circuit 123.

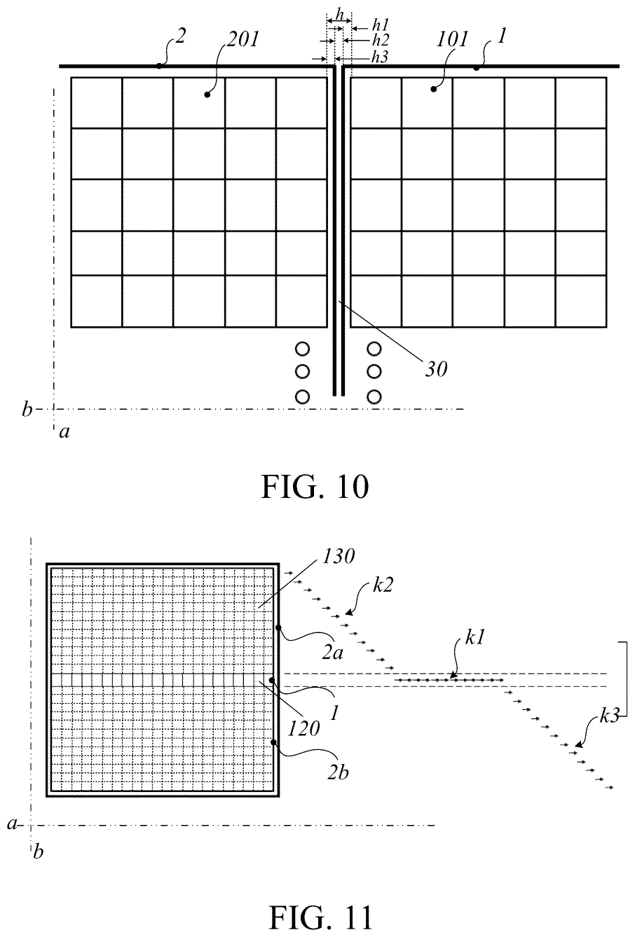

Referring to FIG. 10, each of the first radiation detection surface 10 and the second radiation detection surfaces 20 has a plurality of uniformly arranged pixels (which are used as the smallest image units to detect and receive the radiation). The pixels of the first radiation detection surface 10 can be denoted as photon-counting detection pixels 101, and the pixels of the second radiation detection surface 20 can be denoted as energy-integrating detection pixels 201. The photon-counting detection pixels 101 are located on the photoelectric conversion substrate 121, and the energy-integrating detection pixels 201 are located on the energy-integrating substrate 21. The photon-counting detection module 1 and the energy-integrating detection module 2 are connected by splicing, and a splicing seam 30 is formed between them. The splicing seam 30 between the first radiation detection surface 10 and second radiation detection surfaces 20, which are coplanar, has a splicing gap h along the second direction b. When each of the first radiation detection surface 10 and the second radiation detection surface 20 has a plurality of uniformly arranged pixels, the splicing gap h can be further regarded as the distance along the second direction b between the photon-counting detection pixel 101 closest to the energy-integrating detection module 2 and the energy-integrating detection pixel 201 closest to the photon-counting detection module. The splicing gap h is less than or equal to the size of two pixels. The size of a pixel refers to the width of the pixel along the second direction b. The size of the pixel is generally in the range from 100 microns to 150 microns. The splicing gap h refers to the sum of the distance h1 from the photon-counting detection pixel 101 to an edge of the photoelectric conversion substrate 121, the spacing h2 between the photoelectric conversion substrate 121 and the energy-integrating substrate 21, and the distance h3 from the energy-integrating detection pixel 201 to an edge of the energy-integrating substrate 21 along the second direction b, that is, h=h1+h2+h3. In some embodiments, h is equal to the size of one or two pixels. For example, the values of h1 and h3 can be set first, and then the value of the spacing h2 can be adjusted by mechanical adjustment so that h is equal to the size of one or two pixels. In this case, a bad line correction algorithm can be directly used to correct the image data of the hybrid flat panel detector 100 to eliminate the impact of the splicing gap on a generated image. In other embodiments, h is smaller than the size of one pixel. In this case, the impact of the splicing gap on the generated image can be ignored.

As shown in FIG. 10, in this embodiment, the splicing of the photon-counting detection module and the energy-integrating detection modules is realized by directly splicing the photoelectric conversion substrate 121 of the photon-counting detection module with the energy-integrating substrate 21 of the energy-integrating detection modules, that is, the detection surface of the hybrid flat panel detector of the present disclosure can be formed by splicing the borderless photoelectric conversion substrate 121 with the borderless energy-integrating substrate 21. The spacing between the photoelectric conversion substrate 121 and the energy-integrating substrate 21 can be preset by means of mechanical fixing. For example, the spacing h2 between the photoelectric conversion substrate 121 and the energy-integrating substrate 21 is preset to be less than half of the size of the pixel. In addition, the distance h1 from the photon-counting detection pixel 101 to the edge of the photoelectric conversion substrate 121 is preset to be less than half of the size of the pixel, and the distance h3 from the energy-integrating detection pixel 201 to the edge of the energy-integrating substrate 21 is preset to be less than half of the size of the pixel. As such, the splicing gap h is preset to be less than the size of two pixels. During use, the spacing between the photoelectric conversion substrate 121 and the energy-integrating substrate 21 can be adjusted by mechanical means to adjust the splicing gap h, so that the splicing gap h is, for example, the size of one or two pixels.

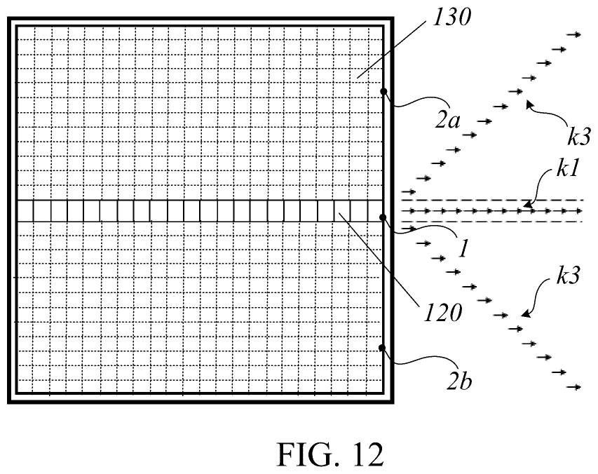

An image data reading method is also provided in an embodiment of the present disclosure. The image data reading method is applied to the above photon-counting-based hybrid flat panel detector 100. Referring to FIG. 11, the first radiation detection surface of the photon-counting detection module 1 includes a plurality of reference photon-counting detection surfaces 120 arranged in sequence along the first direction a. The second radiation detection surfaces of the energy-integrating detection modules 2a and 2b each include an array of reference energy-integrating detection surfaces. The array of the reference energy-integrating detection surfaces includes a plurality of rows of the reference energy-integrating detection surfaces arranged in sequence along the second direction b. Each row of the reference energy-integrating detection surfaces includes a plurality of the reference energy-integrating detection surfaces 130 arranged in sequence along the first direction a.

According to the image data reading method of the present disclosure, the modes for reading data from the hybrid flat panel detector 100 include a first data reading mode k1, a second data reading mode k2, and a third data reading mode k3. Referring to FIG. 11, the first data reading mode k1 is configured to read the image data of each reference photon-counting detection surface 120 in sequence along the first direction a. The second data reading mode k2 is configured to read the image data of each reference energy-integrating detection surface 130 in the energy-integrating detection module 2 row by row in the direction along the second direction b and close to the photon-counting detection module 1 (the rows are along the direction a). The third data reading mode k3 is configured to read the image data of each reference energy-integrating detection surface 130 in the energy-integrating detection module 2 row by row in the direction along the second direction b and away from the photon-counting detection module 1 (the rows are along the direction a). The image data reading method includes at least one of Step A and Step B described below.

Referring to FIG. 11, in this embodiment, the image data reading method includes Step A: an image data reading direction is consistent with the arrangement direction of the energy-integrating detection modules 2a and 2b and the photon-counting detection module 1 (i.e., the second direction b), that is, the image data acquired by the energy-integrating detection module 2a, the photon-counting detection module 1, and the energy-integrating detection module 2b are read in sequence according to the arrangement direction of the energy-integrating detection modules 2a, 2b and the photon-counting detection module 1. For example, referring to FIG. 11, for the energy-integrating detection module 2a, the photon-counting detection module 1, and the energy-integrating detection module 2b, which are arranged in sequence along the second direction b, the image data of the energy-integrating detection module 2a is first read in the second data reading mode k2, then the image data of the photon-counting detection module 1 is read in the first data reading mode k1, and finally the image data of the energy-integrating detection module 2b is read in the third data reading mode k3.

Referring to FIG. 12, in this embodiment, the image data reading method includes Step B: the image data of the energy-integrating detection modules 2a and 2b and the photon-counting detection module 1 are read synchronously. For example, referring to FIG. 12, for the energy-integrating detection module 2a, the photon-counting detection module 1, and the energy-integrating detection module 2b, which are arranged in sequence along the second direction b, the image data of the photon-counting detection module 1 and the two energy-integrating detection modules 2a and 2b are read synchronously, that is, the image data of the photon-counting detection module 1 is read in the first data reading mode k1, and the image data of the two energy-integrating detection modules 2a and 2b are read synchronously in the third data reading mode k3. As such, data can be read quickly. In other embodiments, while the image data of the photon-counting detection module 1 is read in the first data reading mode k1, the image data of the two energy-integrating detection modules 2a and 2b can also be read synchronously in the second data reading mode k2.

The energy-integrating detection module 2a, the photon-counting detection module 1, and the energy-integrating detection module 2b, which are arranged in sequence along the second direction b, can be regarded as a group of detection modules. For reading the data of the group of detection modules, either Step A or Step B can be used. For multiple groups of detection modules formed by a plurality of photon-counting detection modules 1 and a plurality of energy-integrating detection modules 2, if there is no shared energy-integrating detection module 2 between two or more groups of detection modules whose data needs to be read, each group of detection modules can choose Step A or Step B to read data arbitrarily. If there is a common energy-integrating detection module 2 between two groups of detection modules whose data needs to be read, Step A can be used to read the data of each group of detection modules in sequence.

Certainly, in this embodiment, one of the above data reading modes can also be selected to read the data of the corresponding detection module. For example, if only high-quality photon-counting medical images are needed during clinical diagnosis, only the data of the photon-counting detection module 1 can be read in the first data reading mode k1, and the data of the energy-integrating detection modules 2 can be omitted.

Based on the above image data reading method, a non-transitory computer-readable storage medium is provided in an embodiment, storing a program executable by a processor. The processor is configured to read the image data from the above photon-counting-based hybrid flat panel detector 100. With reference to the embodiments described above for the image data reading method, the modes for reading the data from the hybrid flat panel detector 100 include the first data reading mode k1, the second data reading mode k2, and the third data reading mode k3. The first data reading mode k1 is configured to read the image data of each reference photon-counting detection surface 120 in sequence along the first direction a. The second data reading mode k2 is configured to read the image data of each reference energy-integrating detection surface 130 in the energy-integrating detection module 2 row by row in the direction along the second direction b and close to the photon-counting detection module 1 (the rows are along the direction a). The third data reading mode k3 is configured to read the image data of each reference energy-integrating detection surface 130 in the energy-integrating detection module 2 row by row in the direction along the second direction b and away from the photon-counting detection module 1 (the rows are along the direction a).

When the program is executed by the processor, the processor implements at least one of Step A and Step B.

Referring to FIG. 11, in Step A, for the energy-integrating detection module 2a, the photon-counting detection module 1, and the energy-integrating detection module 2b, which are arranged in sequence along the second direction b, the image data of the energy-integrating detection module 2a is first read in the second data reading mode k2, then the image data of the photon-counting detection module 1 is read in the first data reading mode k1, and finally the image data of the energy-integrating detection module 2b is read in the third data reading mode k3.

Referring to FIG. 12, in Step B, for the energy-integrating detection module 2a, the photon-counting detection module 1, and the energy-integrating detection module 2b, which are arranged in sequence along the second direction b, the image data of the photon-counting detection module 1 and the two energy-integrating detection modules 2a and 2b are read synchronously, that is, the image data of the photon-counting detection module 1 is read in the first data reading mode k1, and the image data of the two energy-integrating detection modules 2a and 2b are read synchronously in the third data reading mode k3. In other embodiments, while the image data of the photon-counting detection module 1 is read in the first data reading mode k1, the image data of the two energy-integrating detection modules 2a and 2b can also be read synchronously in the second data reading mode K2.

The energy-integrating detection module 2a, the photon-counting detection module 1, and the energy-integrating detection module 2b, which are arranged in sequence along the second direction b, can be regarded as a group of detection modules. For reading the data of the group of detection modules, either Step A or Step B can be used. For multiple groups of detection modules formed by a plurality of photon-counting detection modules 1 and a plurality of energy-integrating detection modules 2, if there are no shared energy-integrating detection module 2 between two or more groups of detection modules whose data needs to be read, each group of detection modules can choose Step A or Step B to read data arbitrarily. If there is a shared energy-integrating detection module 2 between two groups of detection modules whose data needs to be read, Step A can be used to read the data of each group of detection modules in sequence.

In this embodiment, the processor can also select one of the above data reading modes to read the data of the corresponding detection module. For example, if only the high-quality photon-counting medical images are needed during the clinical diagnosis, only the data of the photon-counting detection module 1 can be read in the first data reading mode k1, and the data of the energy-integrating detection modules 2 can be omitted.

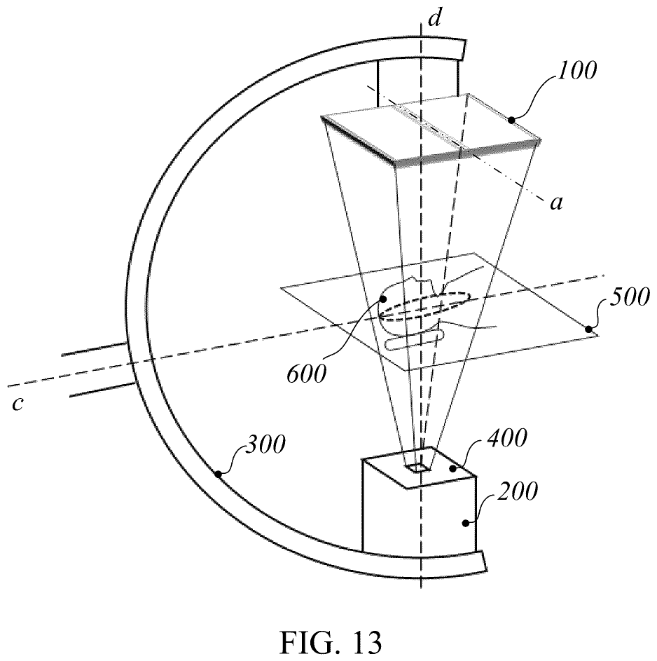

In an embodiment, referring to FIG. 13, the photon-counting-based hybrid flat panel detector 100 is coupled to an end of the C-shaped arm 300. The photon-counting-based hybrid flat panel detector 100 can rotate with the rotation of the C-shaped arm around the first reference line c to scan the subject. The data reading modes of the photon-counting-based hybrid flat panel detector 100 further include a fourth data reading mode k4. The fourth data reading mode k4 is a hybrid data reading mode. In the fourth data reading mode k4, during each scan, photon-based image data obtained by scanning with the photon-counting detection units 12 in the photon-counting-based hybrid flat panel detector 100 can be used to enhance energy-integrating-based image data obtained by scanning with the energy-integrating detection modules 2 in the photon-counting-based hybrid flat panel detector 100. For example, based on a machine learning algorithm, the photon-based image data can be used to perform image enhancement on the energy-integrating-based image data, so as to improve the imaging quality when performing 2D imaging or 3D imaging based on the image data obtained from the scan.

The above computer-readable storage medium may be, for example, but not limited to, an electrical, magnetic, optical, electromagnetic, infrared, or semiconductor system, apparatus, or device, or any combination thereof. More specific examples of the computer-readable storage medium may include, but are not limited to, a portable computer disk, a hard disk, a Random Access Memory (RAM), a Read-Only Memory (ROM), an Erasable Programmable Read-Only Memory (EPROM or flash memory), an optical fiber, a portable Compact Disc Read-Only Memory (CD-ROM), an optical storage device, a magnetic storage device, or any suitable combination of the above. In the present disclosure, the computer-readable storage medium may be any tangible medium that contains or stores a program, and the program may be used by or in combination with an instruction execution system, apparatus, or device. In the present disclosure, a computer-readable signal medium may include a data signal propagated in baseband or as part of a carrier wave, in which computer-readable program instructions are carried. Such a propagated data signal may take various forms, including but not limited to an electromagnetic signal, an optical signal, or any suitable combination thereof. The computer-readable signal medium may also be any computer-readable medium other than the computer-readable storage medium. The computer-readable signal medium may send, propagate, or transmit a program for use by or in combination with an instruction execution system, apparatus, or device. The program instruction contained on the computer-readable medium may be transmitted by any appropriate medium, including but not limited to wires, optical cables, Radio Frequency (RF), etc., or any suitable combination thereof.

In a possible implementation, the image data reading method of the present disclosure may also be implemented in the form of a program product. The program product includes program instructions. When the program product runs on a terminal device, the program instructions, when executed, enable the terminal device to implement the image data reading method described in the above embodiments. The program instructions for executing the method provided in the present disclosure may be written in any combination of one or more programming languages. The program instructions may be executed entirely on a user's device, partially on the user's device, executed as an independent software package, partially on the user's device and partially on a remote device, or entirely on the remote device.

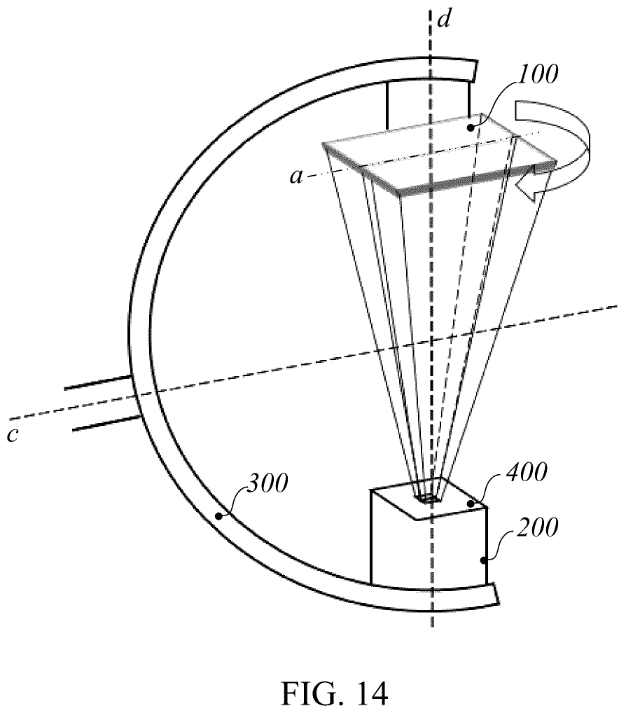

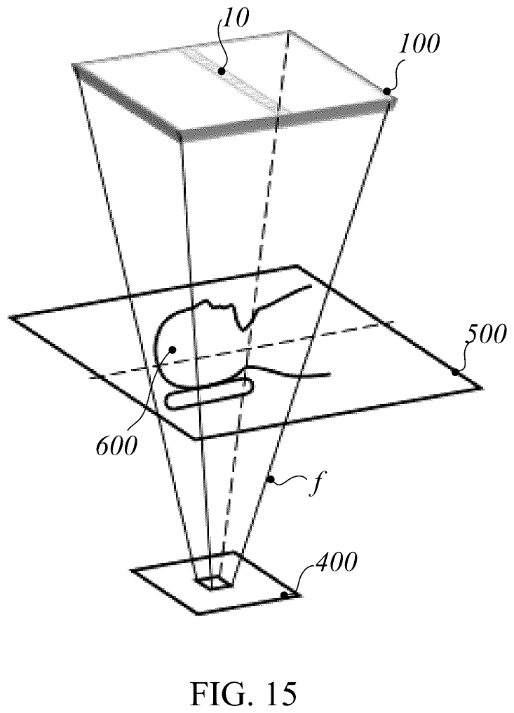

Referring to FIG. 13 and FIG. 14, based on the above photon-counting-based hybrid flat panel detector 100, a medical imaging apparatus is further provided in this embodiment. The medical imaging apparatus includes a C-shaped arm 300, a radiation source 200, the above hybrid flat panel detector 100, and a collimator 400. The C-shaped arm 300 has opposite two ends. The radiation source 200 is disposed on one of the two ends of the C-shaped arm 300, and the radiation source 200 is configured to emit the radiation, such as X-rays. The hybrid flat panel detector 100 is disposed on the other end of the C-shaped arm 300, and the first radiation detection surface 10 and the second radiation detection surfaces 20 face the radiation source 200. Moreover, the first radiation detection surface 10 and the second radiation detection surfaces 20 are perpendicular to a reference line penetrating the two ends of the C-shaped arm 300. The first radiation detection surface 10 and the second radiation detection surfaces 20 can detect the radiation emitted by the radiation source 200. The collimator 400 is disposed on the radiation source 200, and can limit the radiation emitted by the radiation source 200 to a beam of a predetermined shape, such as a cone beam f or a fan beam g. The C-shaped arm 300 is defined to have a first reference line c and a second reference line d. The second reference line d is the reference line penetrating the two ends of the C-shaped arm 300. The first reference line c is parallel to a plane where the C-shaped arm 300 is located and perpendicular to the second reference line d. The medical imaging apparatus further includes an examination table 500 for carrying a patient 600. The second reference line d is perpendicular to a surface of the examination table 500. The examination table 500 can move relative to the C-shaped arm 300 along the direction of the first reference line c, so that the patient 600 enters the detection space defined by the C-shaped arm 300. Thus, medical imaging examination can be completed with the cooperation of the radiation source 200, the collimator 400, and the hybrid flat panel detector 100. During the medical imaging examination, the examination table 500 can move along the direction of the first reference line c, or along the direction perpendicular to the first reference line c and the second reference line d, or along other directions, to facilitate the medical imaging apparatus to image different parts of the body of the patient 600.

In some embodiments, after the hybrid flat panel detector 100 is arranged on the C-shaped arm 300, the initial position of the hybrid flat panel detector 100 can be set such that the length direction of the first radiation detection surface 10 (that is, the first direction a) is perpendicular to the plane defined by the C-shaped arm 300 (as shown in FIG. 13), or such that the first direction a is parallel to the plane defined by the C-shaped arm 300 (as shown in FIG. 14). In other embodiments, the initial position of the hybrid flat panel detector 100 can also be set such that the first direction a is arranged at an angle to the plane where the C-shaped arm 300 is located. During the operation of the medical imaging apparatus, the hybrid flat panel detector 100 can rotate around the second reference line d. The C-shaped arm 300 can rotate around the first reference line c, and the C-shaped arm 300 can also rotate the hybrid flat panel detector around the second reference line d. The rotation of at least one of the hybrid flat panel detector 100 and the C-shaped arm 300, and/or the movement of the examination table 500 can realize the imaging of the different parts of the body of the patient by the medical imaging apparatus.

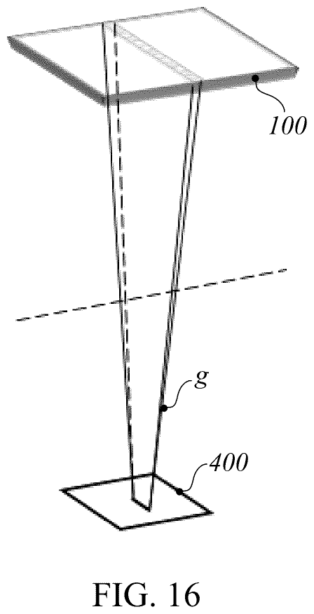

As shown in FIG. 15 and FIG. 16, the medical imaging apparatus of this embodiment can perform 2D imaging, including 2D full-frame imaging and 2D strip imaging. Specifically, in an embodiment, referring to FIG. 15, the collimator 400 limits the shape of the radiation into a cone beam f. The radiation of the cone beam f is attenuated after passing through the patient 600 and is received by the first radiation detection surface 10 and the second radiation detection surfaces 20. Based on the image data generated by the photon-counting detection module 1 and the energy-integrating detection modules 2, a full-frame image can be obtained. The full-frame image includes a full-frame image with strip-shaped high-performance information (based on the image data of the photon-counting detection module 1 and the energy-integrating detection modules 2) and an ordinary full-frame image (based on the image data of the energy-integrating detection modules 2). The ordinary full-frame image can also be directly obtained by removing an energy level of the strip-shaped high-performance information and merging thereby. In another embodiment, referring to FIG. 16, the collimator 400 limits the shape of the radiation to a fan beam g. The extending direction of the first radiation detection surface 10 is parallel to the fan beam g. The radiation of the fan beam g can be detected and received by the first radiation detection surface 10, so as to achieve photon-counting strip imaging.

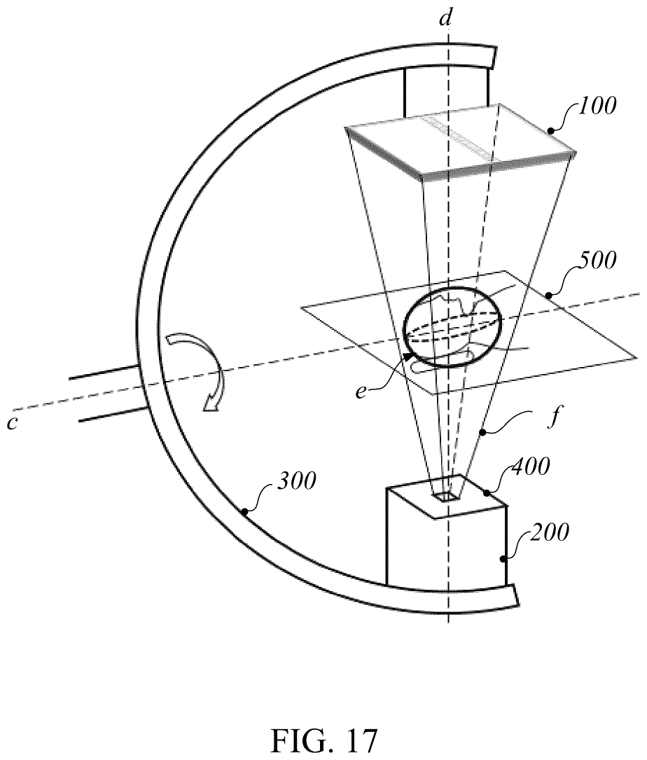

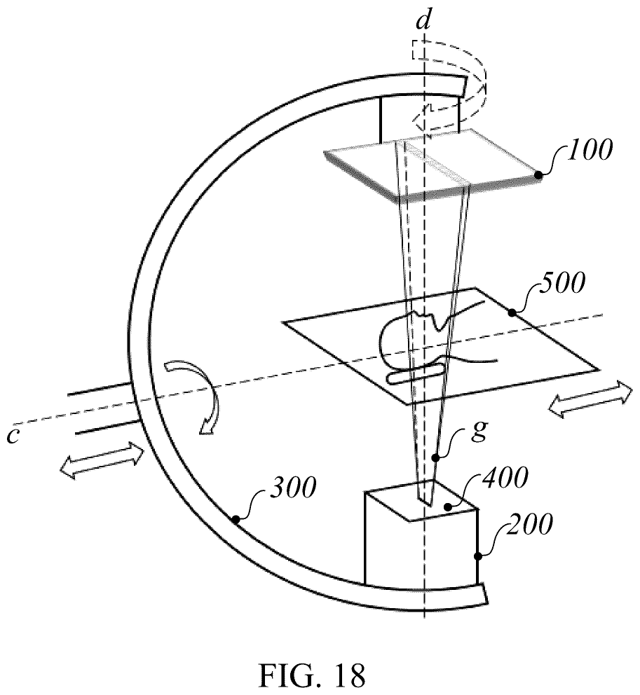

The medical imaging apparatus can perform 3D imaging. In an embodiment, referring to FIG. 17, the C-shaped arm 300 rotates around the first reference line c. After obtaining a certain number of full-frame images, Cone Beam Computed Tomography (CBCT) reconstruction is performed, so that a 3D image can be constructed for a CBCT three-dimensional imaging area e. The 3D image includes a three-dimensional annular image with photon-counting information generated based on the image data of the photon-counting detection module 1. The acquisition of the full-frame image can refer to the content described above with reference to FIG. 15, which is not repeated here. In another embodiment, referring to FIG. 18, the C-shaped arm 300 rotates around the first reference line c to obtain a circle of strip images. At least one of the examination table 500 and the C-shaped arm 300 is controlled to move, to change the position of the examination table 500 relative to the hybrid flat panel detector 100 along the direction of the first reference line c. The scanning is repeated to obtain strip images at the next position, and CT reconstruction is performed to obtain a photon-counting CT three-dimensional imaging result. In yet another embodiment, referring to FIG. 18, at least one of the C-shaped arm 300, the examination table 500, and the hybrid flat panel detector 100 rotates by multiple angles around the second reference line d. The 3D imaging is performed at each angle in the aforementioned manner, so as to obtain a multi-angle 3D imaging result.

Although the present disclosure is disclosed above with some embodiments, the above embodiments are not intended to limit the technical solution of the present disclosure. For any person skilled in the art, without departing from the scope of the technical solution of the present disclosure, many possible variations and modifications can be made to the technical solution of the present disclosure by using the technical content disclosed above, or modified into equivalent embodiments. Therefore, any simple change, equivalent variation, or modification made to the above embodiments according to the technical essence of the present disclosure without departing from the content of the technical solution of the present disclosure still falls within the scope of protection of the technical solution of the present disclosure.

What is claimed is:

1. A photon-counting-based hybrid flat panel detector, comprising:
a photon-counting detection module having a first radiation detection surface, wherein the first radiation detection surface extends in a strip shape along a first direction; and
two energy-integrating detection modules each having a planar second radiation detection surface;
wherein the two energy-integrating detection modules are arranged on opposite two sides of the photon-counting detection module along a second direction, respectively, so that the second radiation detection surfaces are provided on the opposite two sides of the first radiation detection surface along the second direction, respectively, the second direction being parallel to the first radiation detection surface and perpendicular to the first direction; and
the first radiation detection surface and the second radiation detection surfaces jointly form a radiation detection surface of the hybrid flat panel detector.

2. The photon-counting-based hybrid flat panel detector according to claim 1, wherein the first radiation detection surface is located at a central position of the radiation detection surface of the photon-counting-based hybrid flat panel detector along the second direction.

3. The photon-counting-based hybrid flat panel detector according to claim 1, wherein the first radiation detection surface penetrates the radiation detection surface of the photon-counting-based hybrid flat panel detector along the first direction.

4. The photon-counting-based hybrid flat panel detector according to claim 1, wherein the first radiation detection surface is coplanar with the second radiation detection surface of each of the two energy-integrating detection modules.

5. The photon-counting-based hybrid flat panel detector according to claim 1, wherein the second radiation detection surface of one of the two energy-integrating detection modules is coplanar with the second radiation detection surface of the other one of the two energy-integrating detection modules, and is non-coplanar with the first radiation detection surface.

6. The photon-counting-based hybrid flat panel detector according to claim 5, wherein an edge of the second radiation detection surface close to the first radiation detection surface is arc-shaped.

7. The photon-counting-based hybrid flat panel detector according to claim 1, wherein the photon-counting detection module comprises a main photon detection circuit and at least one photon-counting detection unit, the photon-counting detection unit is electrically connected to the main photon detection circuit, and the photon-counting detection unit has a reference photon-counting detection surface.

8. The photon-counting-based hybrid flat panel detector according to claim 7, wherein the at least one photon-counting detection unit comprises a plurality of photon-counting detection units arranged in at least one row, each of the plurality of photon-counting detection units has a reference photon-counting detection surface, and all the reference photon-counting detection surfaces are coplanar to form the first radiation detection surface.

9. The photon-counting-based hybrid flat panel detector according to claim 8, wherein the plurality of photon-counting detection units are arranged along the first direction.

10. The photon-counting-based hybrid flat panel detector according to claim 7, wherein the photon-counting detection unit comprises:
a photoelectric conversion substrate, an end surface thereof being configured as the reference photon-counting detection surface;
an electrical signal acquisition substrate, electrically connected to the photoelectric conversion substrate; and
a photon-counting detection circuit, electrically connected to the electrical signal acquisition substrate and the main photon detection circuit.

11. The photon-counting-based hybrid flat panel detector according to claim 1, wherein each of the two energy-integrating detection modules comprises an energy-integrating substrate and an energy-integrating detection circuit electrically connected to the energy-integrating substrate, and an end surface of the energy-integrating substrate is configured as the second radiation detection surface.

12. The photon-counting-based hybrid flat panel detector according to claim 4, wherein the first radiation detection surface of the photon-counting detection module and the second radiation detection surfaces of the two energy-integrating detection modules are spliced together to form the radiation detection surface of the photon-counting-based hybrid flat panel detector.

13. The photon-counting-based hybrid flat panel detector according to claim 12, wherein each of the first radiation detection surface and the second radiation detection surfaces has a pixel array, and each pixel array comprises a plurality of uniformly arranged pixels; for the adjacent first radiation detection surface and second radiation detection surface, a splicing gap along the second direction between a column of pixels of the first radiation detection surface closest to the second radiation detection surface and a column of pixels of the second radiation detection surface closest to the first radiation detection surface is less than or equal to a size of two pixels.

14. The photon-counting-based hybrid flat panel detector according to claim 13, wherein the splicing gap is equal to a size of one pixel.

15. The photon-counting-based hybrid flat panel detector according to claim 1, wherein an area of each of the second radiation detection surfaces is larger than that of the first radiation detection surface.

16. An image data reading method, applied to the photon-counting-based hybrid flat panel detector according to claim 1, wherein the first radiation detection surface comprises a plurality of reference photon-counting detection surfaces arranged in sequence along the first direction, the two energy-integrating detection modules comprise a first energy-integrating detection module and a second energy-integrating detection module, and each of the second radiation detection surfaces comprises a plurality of rows of reference energy-integrating detection surfaces arranged in sequence along the second direction, wherein the image data reading method comprises:
reading image data of the first energy-integrating detection module, the photon-counting detection module, and the second energy-integrating detection module in sequence along the second direction; or
reading the image data of the first energy-integrating detection module, the photon-counting detection module, and the second energy-integrating detection module synchronously.

17. The image data reading method according to claim 16, wherein reading the image data of the photon-counting detection module comprises:
reading the image data of the photon-counting detection module in a first data reading mode, wherein the first data reading mode is configured to read the image data of the reference photon-counting detection surfaces in sequence along the first direction.

18. The image data reading method according to claim 16, wherein reading the image data of the first energy-integrating detection module, the photon-counting detection module, and the second energy-integrating detection module in sequence along the second direction comprises:
reading the image data of the first energy-integrating detection module in a second data reading mode; and
reading the image data of the second energy-integrating detection module in a third data reading mode;
wherein the second data reading mode is configured to read the image data of the reference energy-integrating detection surfaces in the energy-integrating detection module row by row in a direction along the second direction and close to the photon-counting detection module;
wherein the third data reading mode is configured to read the image data of the reference energy-integrating detection surfaces in the energy-integrating detection module row by row in a direction along the second direction and away from the photon-counting detection module.

19. A medical imaging apparatus, comprising:
a C-shaped arm, having opposite two ends;
a radiation source, disposed on one of the two ends of the C-shaped arm;
a collimator, disposed on the radiation source to limit radiation emitted by the radiation source to a beam of a predetermined shape; and
the photon-counting-based hybrid flat panel detector according to claim 1, disposed on the other end of the two ends of the C-shaped arm, wherein the first radiation detection surface and the second radiation detection surfaces face the radiation source to receive the beam.

20. The medical imaging apparatus according to claim 19, wherein the collimator is configured to limit radiation emitted by the radiation source to a fan beam, the first radiation detection surface of the photon-counting-based hybrid flat panel detector is configured to receive the radiation of the fan beam, and an extending direction of the first radiation detection surface is parallel to the fan beam.
